(19) United States
(12) Patent Application Publication
(10) Pub. No.: US 2024/0033211 A1
(43) Pub. Date: Feb. 1, 2024

(54) EXTRACT OF AT LEAST ONE PLANT OF THE CROCUS SATIVUS SPECIES COMPRISING A HIGH CONTENT OF CROCINS AND A LOW CONTENT OF SAFRANAL, AND COSMETIC USE THEREOF AS ANTIOXIDANT

(71) Applicant: L'OREAL, Paris (FR)

(72) Inventors: Christophe RONDOT, Aulnay-Sous-Bois (FR); Saliou NGOM, Aulnay-Sous-Bois (FR)

(21) Appl. No.: 18/256,655

(22) PCT Filed: Dec. 13, 2021
(86) PCT No.: PCT/EP2021/085487

§ 371 Date: Jun. 9, 2023

(30) Foreign Application Priority Data
Dec. 14, 2020 (FR) FR2013205

(51) Int. Cl. A61K 8/9794; A61K 36/88; A61K 8/60; A61Q 19/08; A61K 8/33

(52) U.S. Cl. CPC A61K 8/9794; A61K 36/88; A61K 8/604; A61Q 19/08; A61K 8/33; A61K 2236/53; A61K 2236/51; A61K 2236/333

(57) ABSTRACT

The present application relates to an extract of at least one plant of the Crocus sativus species comprising (i) at least 20% by dry weight of one of more crocin(s) relative to the total weight of the components of the dry extract and (ii) less than 0.08% by dry weight of safranal relative to the total weight of the components of the dry extract, to the process for preparing same, and the use thereof as an antioxidant.

The present invention relates to the field of skincare.

More particularly, the present invention aims to provide a novel active agent used as antioxidant.

The use of antioxidants in cosmetics is very important and increasingly widespread. Thus, antioxidants make it possible to combat the free radicals (O_{2°}·, OH°, ¹O₂, etc.) that notably induce cell ageing.

They may therefore be used in various cosmetic areas such as anti-ageing, protection against oxidative stresses and especially exogenous stresses due to sun exposure or the environment (pollution, smoke), or the prevention of pigmentation (the synthesis of melanin is an oxidative process).

This antioxidant activity is in particular advantageous for limiting the peroxidation of skin surface lipids and in particular photoinduced peroxidation of sebaceous lipids, such as squalene.

It is in fact known that lipids which are at the surface of the skin are continuously subjected to external attacks, and in particular the air, atmospheric pollutants and visible radiation and especially ultraviolet (UV) radiation, and that the most exposed to external attacks are those contained in the fatty secretions of the skin, such as sebum, which is rich in squalene. The presence in the squalene molecules of six double bonds makes these molecules sensitive to oxidation phenomena. Thus, during prolonged exposure to UV radiation, squalene becomes photoperoxidized to give squalene peroxides.

This high production of squalene peroxides causes in particular a series of chain degradations, in particular on the skin, giving rise to numerous skin disorders.

Thus, these squalene peroxides are involved in particular in the premature ageing of the skin as described by Keiko OH Sawa et al. (J. Toxicol. Sc., 1984: 19, pp 151-159).

Many antioxidants exist already, such as tocopherol (vitamin E) or derivatives thereof, vitamin C or derivatives thereof, carotenoids, ubiquinone, green tea, etc.

However, there is an ongoing need for new active agents that are capable of exerting an antioxidant cosmetic action and in particular of combating oxidative stress of the skin.

Surprisingly, the inventors have demonstrated that an extract of at least one plant from the Crocus sativus species comprising (i) at least 20% by dry weight of one of more crocin(s) relative to the total weight of the components of the dry extract and (ii) less than 0.08% by dry weight of safranal relative to the total weight of the components of the dry extract, makes it possible to combat squalene peroxidation, and therefore has an antioxidant activity while having a pleasant odour of low intensity, and is therefore compatible with use within cosmetic products.

An extract of saffron (Crocus sativus) comprising 10% by weight of crocins and having antioxidant properties is known from the prior art CN108542840A, however this extract has a much lower antioxidant activity, in particular regarding its ability to combat squalene peroxidation compared to the extract according to the invention as shown in Example 4 of the present application.

Moreover, an extract of saffron (Crocus sativus) comprising 27% by weight of crocins and 0.098% by weight of safranal is known from the literature (Quality and functionality of saffron quality control, species assortment and affinity of extract and isolated saffron compounds to NMDA and sigma-1 receptors, Matthias Lechtenberg, Dirk Schepmann, Michael Niehues, Nils Hellenbrand, Bernhard Wünsch, Andreas Hensel, published in June 2008 in the journal Planta Medica). However, this extract is not disclosed as having antioxidant properties. Moreover, the safranal content thereof, greater than 0.08%, generates a powerful and unpleasant odour that does not allow use within cosmetic products as shown by Example 5 of the present application.

Thus, a first subject of the present invention is an extract of at least one plant of the Crocus sativus species comprising (i) at least 20% by dry weight of one of more crocin(s) relative to the total weight of the components of the dry extract and (ii) less than 0.08% by dry weight of safranal relative to the total weight of the components of the dry extract.

Another subject of the present invention is a composition comprising said extract in a physiologically acceptable medium.

Another subject of the present invention relates to a cosmetic treatment method comprising the application of said extract or of said composition to keratin materials, notably the skin, to care for the skin, and notably:
to prevent and/or treat skin disorders induced by oxidative stress, notably induced by UV rays and/or pollution, in particular atmospheric pollution;
and/or to prevent and/or treat the signs of skin ageing;
and/or to prevent and/or treat a dull and/or non-uniform appearance of the complexion;
and/or to improve the radiance and/or the uniformity of the complexion;
and/or to prevent pigmentation disorders such as actinic lentigo or post-inflammatory hyperpigmentation.

The present invention also relates to the cosmetic use of said extract to care for the skin, and notably,
to prevent and/or treat skin disorders induced by oxidative stress, notably induced by UV rays and/or pollution, in particular atmospheric pollution;
and/or to prevent and/or treat the signs of skin ageing;
and/or to prevent and/or treat a dull and/or non-uniform appearance of the complexion;
and/or to improve the radiance and/or the uniformity of the complexion;
and/or to prevent pigmentation disorders such as actinic lentigo or post-inflammatory hyperpigmentation.

The present invention also relates to the cosmetic use of said extract as antioxidant agent.

Another subject of the present invention is a process for preparing said extract of at least one plant of the Crocus sativus species according to the invention comprising at least one step of extracting a part of at least one plant of the Crocus sativus species using an aqueous solvent chosen from water or a mixture of water and one or more water-miscible organic solvents, preferably using an ethanol/water mixture in a volume ratio of 1/1.

### Definition

A “keratin material” is understood to mean human skin.

The term “skin” means all of the skin of the body, and the scalp, and preferably the skin of the face, neckline, neck, arms and forearms, or even more preferably still the skin of the face (in particular of the forehead, nose, cheeks and chin), neckline and neck.

According to the invention, the term “preventing” or “prevention” means reducing the risk of occurrence or slowing down the occurrence of a given phenomenon.

The term “treating” or “treatment” means any action that aims to improve the comfort or the well-being of an individual; this term thus equally covers attenuating, relieving and curing.

The term “antioxidant” denotes a compound that decreases or prevents the oxidation of other chemical compounds.

Oxidation forms part of a redox reaction that transfers electrons from a substance to an oxidizing agent. This reaction can produce free radicals. An antioxidant agent may notably be a free-radical scavenger.

For the purposes of the present invention, the term “free radicals” is understood to mean chemical species with one or more unpaired electrons in its outer shell, such as for example free radicals of oxygen.

The oxygen free radicals are notably:
singlet oxygen ·O—O·;
the superoxide radical anion O₂·⁻;
the hydroxyl radical HO·;
the hydroperoxyl radical HO₂·;
the peroxide radical (ROO·) and the alkoxy radical (RO·) where R is a carbon chain.

For the purposes of the present invention, the term “free radical scavenger” is understood to mean a compound which neutralizes the free radicals formed.

For the purposes of the present invention, the term “oxidative stress” covers any damage caused by an increase in oxygen free radicals in a subject.

### DETAILED DESCRIPTION

Extract of Crocus sativus (Saffron)

The present invention relates to an extract of at least one plant of the Crocus sativus species comprising (i) at least 20% by dry weight of one of more crocin(s) relative to the total weight of the components of the dry extract and (ii) less than 0.08% by dry weight of safranal relative to the total weight of the components of the dry extract.

Crocus sativus (Crocus sativus L., 1753) or autumn crocus, saffron crocus, saffron or true saffron is a geophyte plant of the family Iridaceae, from which saffron is extracted. Crocus sativus belongs to a group of ten or so species of Mediterranean crocuses from which saffron has been produced since ancient times.

Crocus sativus is a glabrate, geophyte perennial plant of 10 to 30 cm. Its corm (underground storage stem resembling a bulb) is very sizeable for a crocus (from less than 1 cm in circumference for bulbets to close to 20 cm) and subglobose. The fibrous tissues or layers which surround and protect the corm are composed of fibres crosslinked as long and narrow meshes. The linear leaves appear in autumn, before, with or after the flowers and grow continuously until they enter vegetative rest at the end of spring. They can then measure about 60 cm with a width of a few mm. Numbering from 1 to 10 approximately per bud, they form tufts, initially erect, then drooping, of a beautiful pure green. Each corm produces potentially up to about 10 flowers joined in inflorescence. Stems and spathes remain underground and are therefore invisible. The flowers, organised in ternary symmetry, are spectacular, large, lilac and perfumed. Their perianth is composed of 6 tepals (3 petals and 3 similar sepals) fused in a purplish-blue throat to form the tube of the perianth (mistakenly taken to be the “stem” of the flower). The anthers are bright yellow, 3 in number, longer by half than their filament. The pistil that prolongs the underground ovary, is subdivided into 3 terminal parts, the stigmata. The stigmata of Crocus sativus are hypertrophied, scarlet, odorous, flared in a crenellated trumpet. They often droop out of the perianth under the effect of their size. They are used to produce a rare and precious spice: saffron. In the northern hemisphere, flowering occurs between September and November and lasts from 2 to 6 weeks. Since Crocus sativus is triploid, it does not produce seeds and is therefore exclusively reproduced by the vegetative route.

Saffron, in particular the stigmata, are known for their food use as a condiment.

Saffron is notably native to Italy and the Orient, notably Morocco, but it can also be grown in regions of Greece and Crete, Switzerland, Iran.

In Morocco, the main production sites for saffron correspond to two very localised terroirs of the Siroua Massif, a volcanic massif located at the juncture of the Anti-Atlas and the High-Atlas, the Taliouine terroir, the Tazenakht terroir, which are characterized by a high degree of geographical isolation and by the great dispersion of the growing basins, concentrated around water points. Among the secondary production terroirs, mention may be made of the Ourika terroir.

Advantageously, the Crocus sativus used in the context of the present invention originates from Morocco, such as that originating from the Ourika Valley, in particular located 35 km south-west of Marrakesh, close to Oukaimeden. The Ourika Valley is on the north-west side of the High-Atlas. Harvesting takes place in the autumn period notably from 15 October to 15 November. As an example of saffron (Crocus sativus) originating from the Ourika Valley, mention may notably be made of those originating from the Yves Saint Laurent Gardens, or else those from the Safraniere (Saffron garden) of Ourika.

Preferably, said crocin(s) (i) is(are) present at between 20% and 40% by dry weight relative to the total weight of the components of the dry extract, even more preferentially between 25% and 35% by dry weight relative to the total weight of the components of the dry extract.

Preferably, said safranal (ii) is present at less than 0.07% by dry weight relative to the total weight of the components of the dry extract, even more preferentially at less than 0.06% by dry weight relative to the total weight of the components of the dry extract. In a preferred embodiment, said extract is completely free of safranal i.e. 0%.

The crocin(s) present in said extract is(are) preferably chosen from:
bis-gentiobiosyl-E-crocetin;
gentiobiosyl-glucosyl-E-crocetin;
bis-glucosyl-E-crocetin;
bis-gentiobiosyl-13-Z-crocetin;
gentiobiosyl-glucosyl-13-Z-crocetin;
and mixtures thereof.

Preferably, the crocin(s) is(are) in the form of a mixture comprising:
between 10% and 30% by dry weight of bis-gentiobiosyl-E-crocetin;
between 5% and 10% by dry weight of gentiobiosyl-glucosyl-E-crocetin;
between 0.1% and 5% by dry weight of bis-glucosyl-E-crocetin;
between 0.05% and 2% by dry weight of bis-gentiobiosyl-13-Z-crocetin; relative to the total weight of the components of the dry extract.

Preferably, the crocin(s) is(are) in the form of a mixture comprising:
between 60% and 70% by dry weight of bis-gentiobiosyl-E-crocetin;
between 20% and 30% by dry weight of gentiobiosyl-glucosyl-E-crocetin;
between 5% and 10% by dry weight of bis-glucosyl-E-crocetin;
between 2% and 6% by dry weight of bis-gentiobiosyl-13-Z-crocetin; relative to the total weight of the crocins of the dry extract.

Said extract may also comprise picrocrocin. Picrocrocin may be present in said extract at a concentration of between 10% and 20% by weight relative to the total weight of the components of the dry extract.

Advantageously, said extract is obtained by extraction of a part of at least one plant of the Crocus sativus species using an aqueous solvent. The extraction step can be repeated at least a second time, notably the extraction step can be repeated twice.

The expression “part of a plant of the Crocus sativus species” is understood to mean the flowers, the stems, the leaves, the stigmata, the pistils; preferably the part(s) of plant(s) of the Crocus sativus species from which the extraction is carried out is(are) the pistil(s).

An “aqueous solvent” is understood to mean water or a mixture of water and one or more water-miscible organic solvents. The water-miscible organic solvents can be chosen from linear or branched C1-C6 monoalcohols, such as ethanol, isopropanol or tert-butanol; polyols such as glycerol, propylene glycol, hexylene glycol (or 2-methyl-2,4-pentanediol) and polyethylene glycols; polyol ethers, for instance dipropylene glycol monomethyl ether; and mixtures thereof.

Even more preferably, said extract is obtained by extraction using an ethanol/water mixture in a volume ratio of 1/1.

Advantageously, the extraction is performed for an approximate time ranging from 30 minutes to 8 hours, preferably between 1 and 6 hours, more particularly between 3 and 5 hours such as 4 hours, notably with stirring, for example using an impeller or magnetic stirrer.

The extraction is in particular a maceration at room temperature, for example at 25° C.

Said extract is notably obtained by extraction of from 10 g of pistils of Crocus sativus per litre of solvent to 1000 g of pistils of Crocus sativus per litre of solvent, preferably from 20 g of pistils of Crocus sativus per litre of solvent to 500 g of pistils of Crocus sativus per litre of solvent, better still from 50 g of pistils of Crocus sativus per litre of solvent to 200 g of pistils of Crocus sativus per litre of solvent, for instance 100 g of pistils of Crocus sativus per litre of solvent.

The extract thus obtained can be dried and is then in the form of a dry extract, after removal of one(of the) solvent(s), for example by evaporation of one or more water-miscible organic solvents notably using a rotary evaporator and/or by evaporation of the water notably using a rotary evaporator, by freeze drying, by spray drying, or a combination of these methods.

For the purposes of the present invention, the term “dry extract” means an extract comprising less than 10% by weight of water, preferably less than 7% by weight of water, better still less than 5% or even more preferentially less than 2% by weight of water, or even totally free of water (0%).

In a preferred embodiment, the extract obtained at the end of the extraction step may be subjected to:
i) a step of filtration, in particular on a nylon gauze having a pore size of between 50 μm and 200 μm, and/or
ii) a step of slurrying in cellulose, and/or
iii) one or more clarification steps, preferably the extract obtained on conclusion of the extraction step may be subjected to a first step of clarification on a bed of cellulose and cotton gauze having a pore size of between 5 μm and 15 μm and also a second step of clarification on a cellulose sheet having a pore size of between 0.1 μm and 3 μm, preferably between 0.8 μm and 0.5 μm; and/or
iv) a step of sterilization, notably by passage through a 0.2 μm cartridge.

In a preferred embodiment, each of the abovementioned steps (extraction such as maceration, filtration, slurrying, clarification, evaporation, etc.) are carried out in the absence of light.

Prior to the extraction step, the pistils of Crocus sativus can be dried and/or ground for example in the form of particles with a size of less than or equal to 5 mm, preferably with a size of less than or equal to 2 mm.

The present invention also relates to a process for preparing said extract of at least one plant of the Crocus sativus species according to the invention comprising at least one step of extracting a part of at least one plant of the Crocus sativus species using an aqueous solvent chosen from water or a mixture of water and one or more water-miscible organic solvents, preferably using an ethanol/water mixture in a volume ratio of 1/1.

In this process, the extract obtained at the end of this extraction step may be subjected to the following, preferably consecutive, steps:
i) a step of filtration, in particular on a nylon gauze having a pore size of between 50 μm and 200 μm, and/or
ii) a step of slurrying in cellulose, and/or
iii) one or more clarification steps, preferably the extract obtained on conclusion of the extraction step may be subjected to a first step of clarification on a bed of cellulose and cotton gauze having a pore size of between 5 μm and 15 μm and also a second step of clarification on a cellulose sheet having a pore size of between 0.1 μm and 3 μm, preferably between 0.8 μm and 0.5 μm; and/or
iv) a step of sterilization, notably by passage through a 0.2 μm cartridge.

On conclusion of said extraction step, and optionally of steps i) and/or ii) and/or iii) and/or iv), which are preferably consecutive, said extract may be subjected to a drying step which comprises the removal of one(of the) solvent(s), for example by evaporation of one or more water-miscible organic solvents notably using a rotary evaporator and/or by evaporation of the water notably using a rotary evaporator, by freeze drying, by spray drying, or a combination of these methods.

Composition

The present invention also relates to a composition comprising, in a physiologically acceptable medium, the extract of at least one plant of the Crocus sativus species according to the invention.

Said extract may be present in the composition in a concentration of between 0.0001% and 20% by weight of dry extract, preferably between 0.001% and 10% by weight of dry extract and better still between 0.001% and 5% by weight of dry extract relative to the total weight of the composition.

The term “physiologically acceptable medium” means a medium that is compatible with keratin materials and in particular the skin.

More particularly, said physiologically acceptable medium may comprise water and/or one or more water-miscible organic solvents which may be chosen from linear or branched C1-C6 monoalcohols such as ethanol, isopropanol or tert-butanol; polyols such as glycerol, propylene glycol, hexylene glycol (or 2-methyl-2,4-pentanediol), and polyethylene glycols; polyol ethers such as dipropylene glycol monomethyl ether; and mixtures thereof.

Preferentially, the composition according to the invention has a water content ranging from 20% to 95% by weight, better still from 30% to 70% by weight, relative to the total weight of the composition.

Advantageously, the composition comprises one or more water-miscible organic solvents in a content ranging from 0.5% to 25% by weight, preferably from 5% to 20% by weight, even better still from 10% to 15% by weight relative to the total weight of the composition.

The composition according to the invention may further comprise any adjuvant commonly used in the envisaged application field.

Mention may notably be made of organic solvents, notably C1-C6 alcohols and C2-C10 carboxylic acid esters; carbon-based and/or silicone oils, of mineral, animal and/or plant origin; waxes, pigments, fillers, colorants, surfactants, emulsifiers, coemulsifiers; cosmetic active agents different from the extract according to the invention, UV-screening agents, polymers, hydrophilic or lipophilic gelling agents, thickeners, preserving agents, fragrances, bactericides, odour absorbers and antioxidants.

These optional adjuvants may be present in the composition in a proportion of from 0.001% to 80% by weight, preferably 0.01% to 30% by weight, better still from 0.1% to 20%, relative to the total weight of the composition. Depending on their nature, these adjuvants may be introduced into the fatty phase, into the aqueous phase and/or into the lipid vesicles.

Needless to say, a person skilled in the art will take care to select this or these optional additional ingredients and/or active agents, and/or the amount thereof, such that the advantageous properties of the extract according to the invention are not, or are not substantially, adversely affected by the envisaged addition.

The compositions according to the invention may be in any presentation form conventionally used for topical application and notably in the form of aqueous or aqueous-alcoholic solutions, oil-in-water (O/W), water-in-oil (W/O) or multiple (triple: W/O/W or O/W/O) emulsions, aqueous gels, or dispersions of a fatty phase in an aqueous phase using spherules, these spherules possibly being lipid vesicles of ionic and/or nonionic type (liposomes, niosomes or oleosomes). These compositions are prepared according to the usual methods.

Advantageously, the compositions according to the invention are in the form of a gel, an emulsion, a powder or a paste. In addition, the composition according to the invention may be more or less fluid and may have the appearance of a white or coloured cream, an ointment, a milk, a lotion, a serum, a paste, a foaming gel, a scrub, a mask, a care product, a tonic or a foam. It may optionally be applied to the skin in aerosol form. It may also be in solid form, for example in stick form.

A composition according to the invention may comprise an oily phase.

As oils which may be used in the composition of the invention, mention may for example be made of:
hydrocarbon-based oils,
synthetic esters and ethers, especially of fatty acids, for instance the oils of formulae R′COOR2 and R′COR2 in which R′ represents a fatty acid residue containing from 8 to 29 carbon atoms, and R2 represents a branched or unbranched hydrocarbon-based chain containing from 3 to 30 carbon atoms;
linear or branched hydrocarbons of mineral or synthetic origin;
fatty alcohols having from 8 to 26 carbon atoms;
partially hydrocarbon-based and/or silicone-based fluoro oils;
silicone oils;
mixtures thereof.

In the list of the abovementioned oils, the term “hydrocarbon-based oil” is understood to mean any oil predominantly comprising carbon and hydrogen atoms.

The oily phase may contain other fatty substances that may be present in the oily phase and are, for example, fatty acids including from 8 to 30 carbon atoms, waxes, silicone resins and silicone elastomers. These fatty substances may be chosen in a varied manner by those skilled in the art in order to prepare a composition having the desired properties, for example in terms of consistency or texture.

According to a particular embodiment of the invention, the composition according to the invention is a water-in-oil (W/O) or oil-in-water (O/W) emulsion. The proportion of the oily phase of the emulsion can range from 5% to 80% by weight and preferably from 5% to 60% by weight, with respect to the total weight of the composition. The emulsions generally contain at least one emulsifier chosen from amphoteric, anionic, cationic and nonionic emulsifiers, used alone or as a mixture, and optionally a coemulsifier. The emulsifiers are appropriately chosen according to the emulsion to be obtained (W/O or O/W). The emulsifier and the coemulsifier are generally present in the composition in a proportion ranging from 0.3% to 30% by weight and preferably from 0.5% to 20% by weight relative to the total weight of the composition.

For the W/O emulsions, examples of emulsifiers that may be mentioned include dimethicone copolyols and alkyldimethicone copolyols. A crosslinked elastomeric solid organopolysiloxane including at least one oxyalkylene group may also be used as W/O emulsion surfactant.

For the O/W emulsions, examples of emulsifiers that may be mentioned are nonionic emulsifiers.

The compositions according to the invention may be applied directly to the skin or, alternatively, to cosmetic supports of occlusive or non-occlusive type, intended to be applied locally to the skin. As non-limiting examples of cosmetic supports, mention may be made notably of a patch, a wipe, a roll-on and a pen. The composition may optionally be rinsed off after having been applied to the skin.

Uses and Cosmetic Treatment Method

Another subject of the present invention relates to a cosmetic treatment method comprising the application of said extract or of said composition to keratin materials, notably the skin, to care for the skin, and notably:
to prevent and/or treat skin disorders induced by oxidative stress, notably induced by UV rays and/or pollution, in particular atmospheric pollution;
and/or to prevent and/or treat the signs of skin ageing;
and/or to prevent and/or treat a dull and/or non-uniform appearance of the complexion;
to improve the radiance and/or the uniformity of the complexion;
   to prevent pigmentation disorders such as actinic lentigo or post-inflammatory hyperpigmentation.

The present invention further relates to the cosmetic use of said extract to care for the skin, and notably:
to prevent and/or treat skin disorders induced by oxidative stress, notably induced by UV rays and/or pollution, in particular atmospheric pollution;
and/or to prevent and/or treat the signs of skin ageing;
and/or to prevent and/or treat a dull and/or non-uniform appearance of the complexion;
and/or to improve the radiance and/or the uniformity of the complexion;
and/or to prevent pigmentation disorders such as actinic lentigo or post-inflammatory hyperpigmentation.

The present invention also relates to the cosmetic use of said extract as antioxidant agent.

Among the skin disorders induced by oxidative stress, notably induced by UV rays and/or pollution, in particular atmospheric pollution, mention may notably be made of signs of skin ageing.

Among the signs of skin ageing, mention may notably be made of a thinning of the skin, a loss of firmness, a loss of elasticity, a loss of density or a loss of tonicity of the skin, a deterioration of the surface appearance of the skin, the appearance of a marked microrelief of the skin, the appearance of roughness, the formation and/or presence of fine lines and/or of wrinkles, a deterioration of the radiance of the skin complexion, a papery appearance of the skin, a sagging of the skin, or a withering of the skin.

Throughout the description, including the claims, the expression “comprising a” should be understood as being synonymous with “comprising at least one”, unless otherwise specified.

In addition, the term “at least one” should be understood as being synonymous with “one or more”, unless otherwise specified.

The terms “more than”, “between . . . and . . . ” and “ranging from . . . to . . . ” should be understood as being limits inclusive, unless otherwise specified.

The examples and figures that follow are presented as nonlimiting illustrations of the invention. The compounds are, depending on the case, cited as the chemical names or as the CTFA (International Cosmetic Ingredient Dictionary and Handbook) names.

### EXAMPLES

### Example 1—Preparation of an Extract of Pistils of Crocus sativus Originating from Ourika Valley Comprising 29% of Crocins and 0% of Safranal (According to the Invention)

Saffron, in the form of dried whole pistils of Crocus Sativus flowers originating from Ourika Valley, harvested in 2019, is ground with a cutting mill on a 1 mm screen. 1.9 kg of the orangey powder thus obtained is extracted under stirring in the absence of light with 19 litres of an ethanol-water mixture (1/1) at 25° C. for 4 h. The mixture is prefiltered on a 195 μm nylon gauze in a closed filter. The spent biomass is emptied. The prefiltrate, re-slurried in cellulose (Vitacel LC200) as adjuvant (pre-coat), is pre-clarified on a bed of cellulose in a closed filter equipped with a 10 μm cotton gauze, then is clarified in series in a bell-shaped filter equipped with a 0.8-0.5 μm cellulose sheet, before finally being sterilized on a 0.2 μm cartridge. The aqueous-alcoholic filtrate is then evaporated under vacuum to remove the ethanol, and the aqueous solution with a dry extract of around 10% is spray-dried to obtain the saffron extract in the form of a fine, orange-red powder.

Equipment:
Grinding: Retsch SM100 cutting mill
Extraction and pre-coat: VSA 40E reactor
Pre-filtration: 400 mm closed filter with 195 μm Nylon gauze+50 L mobile receiver
Pre-clarification: De Dietrich agitated filter with 10 μm cotton gauze
Clarification: 400 mm bell-shaped filter with FGC15 sheet
Sterilization: housing with 0.2 μm Millipore filter cartridge
Concentrating: VSA40E reactor
Spray drying: Büchi Mini Spray Dryer B290

The specifications of the extract thus obtained are presented in Table 1 below.

TABLE 1

| | |
|---|---|
| bis-gentiobiosyl-E-crocetin/gentiobiosyl- | 62.4/27/6.9/3.9 |
| glucosyl-E-crocetin/bis-glucosyl-E- | (UV detection λ = |
| crocetin/bis-gentiobiosyl-13-Z-crocetin ratio | 440 nm) |
| Content of total crocin (expressed as bis- | 29.2% |
| gentiobiosyl-E-crocetin TC4) | |
| Content of bis-gentiobiosyl-E-crocetin (TC4) | 18.6% |
| Content of gentiobiosyl-glucosyl-E-crocetin | 7.9% |
| TC3 (expressed as bis-gentiobiosyl-E- | |
| crocetin TC4) | |
| Content of bis-glucosyl-E-crocetin TC2 | 1.8% |
| (expressed as bis-gentiobiosyl-E-crocetin | |
| TC4) | |
| Content of bis-gentiobiosyl-13-Z-crocetin | 0.9% |
| CC4 (expressed as bis-gentiobiosyl-E- | |
| crocetin TC4) | |
| Safranal content | 0% |
| Picrocrocin content | 13.5% |
| Content of total polyphenols (expressed as | 5.2% |
| gallic acid equivalent) | |
| Water content | 4.7% |

TC4: trans-crocin-4.

TC3: trans-crocin-3.

TC2: trans-crocin-2.

CC4: cis-crocin-4.

Content of total crocin (HPLC/DAD, λ=440 nm)

The quantification of the total crocin and of the various crocins (TC4+TC3+TC2+CC4) was carried out by external calibration relative to trans-crocin-4.

The results are therefore expressed as trans-crocin-4. trans-crocin-4 standard: Chengdu Biopurify Phytochemicals Ltd, ref. BP0406 batch PRF10071003, purity 98%. The linearity of the trans-crocin-4 was verified between 0.5 and 100 μg/mL.

The content of total crocin in the extract thus obtained is 29.2% (expressed as trans-crocin-4).

Picrocrocin content (HPLC-CAD countergradient)

The content of picrocrocin was obtained by external calibration from the trans-crocin-4 standard used to quantify the content of crocins.

The content of picrocrocin in the extract thus obtained is 13.5%.

Safranal content (HPLC/DAD, λ=314 nm)

The content of safranal in the extract thus obtained is 0% (not detected).

Content of Total Polyphenols

The content of total polyphenols was obtained by the Folin spectrophotometric method. It is 5.2 g/100 g expressed as gallic acid equivalent.

### Example 2—Preparation of an Extract of Pistils of Crocus sativus Originating from Ourika Valley Comprising 22.7% of Crocins and 0.06% of Safranal (According to the Invention)

Saffron, in the form of dried whole pistils of Crocus Sativus flowers originating from Ourika Valley, harvested in 2018, is ground with a cutting mill on a 2 mm screen. 93 g of the orangey powder thus obtained is extracted under stirring in the absence of light with 0.930 litres of an ethanol-water mixture (1/1) at 21° C. for 4 h. The mixture is prefiltered on a 50 μm nylon gauze on a porcelain Büchner funnel. The spent biomass is emptied, re-extracted and pre-filtered according to the same protocol. The pre-filtrates from the two macerations are combined, clarified on a porcelain Büchner funnel equipped with a 2.5 μm EATON BECO KD3 cellulose sheet. The aqueous-alcoholic filtrate is then evaporated under vacuum to remove the solvents, then freeze-dried to obtain the saffron extract in the form of a fine, bright red powder.

Equipment:

Grinding: IKA® All Basic cutting mill

Extraction: 2.5 L RADLEY reactor

Pre-filtration: porcelain Büchner funnel with 50 μm Nylon gauze.

Clarification: porcelain Büchner funnel with 2.5 μm EATON BECO KD3 sheet.

Concentration: Buchi Rotavapor R215 rotary evaporator equipped with a Buchi Heating Bath B-491 bath, and also with a Vacuubrand PC 3001 VARIO Pro vacuum pump.

Freeze-drying: Labconco FreeZone 4.5 Plus freeze dryer, coupled to a Vaccubrand RZ-6 vacuum pump.

The specifications of the extract thus obtained are presented in Table 2 below.

TABLE 2

| | |
|---|---|
| bis-gentiobiosyl-E-crocetin/gentiobiosyl- | 61/27/7.2/4.8 |
| glucosyl-E-crocetin/bis-glucosyl-E- | (UV detection λ = |
| crocetin/bis-gentiobiosyl-13-Z-crocetin ratio | 440 nm) |
| Content of total crocin (expressed as bis- | 22.7% |
| gentiobiosyl-E-crocetin TC4) | |
| Content of bis-gentiobiosyl-E-crocetin (TC4) | 14.1% |
| Content of gentiobiosyl-glucosyl-E-crocetin | 6.1% |
| TC3 (expressed as bis-gentiobiosyl-E- | |
| crocetin TC4) | |
| Content of bis-glucosyl-E-crocetin TC2 | 1.5% |
| (expressed as bis-gentiobiosyl-E-crocetin | |
| TC4) | |
| Content of bis-gentiobiosyl-13-Z-crocetin | 1.0% |
| CC4 (expressed as bis-gentiobiosyl-E- | |
| crocetin TC4) | |
| Safranal content | 0.06% |
| Picrocrocin content | 13.5% |
| Content of total polyphenols (expressed as | 4.6% |
| gallic acid equivalent) | |

TC4: trans-crocin-4.

TC3: trans-crocin-3.

TC2: trans-crocin-2.

CC4: cis-crocin-4.

Content of total crocin (HPLC/DAD, λ=440 nm)

The quantification of the total crocin and of the various crocins (TC4+TC3+TC2+CC4) was carried out by external calibration relative to trans-crocin-4.

The results are therefore expressed as trans-crocin-4. trans-crocin-4 standard: Chengdu Biopurify Phytochemicals Ltd, ref. BP0406, purity 98%. The linearity of the trans-crocin-4 was verified between 0.5 and 100 μg/mL.

The content of total crocin in the extract thus obtained is 22.7% (expressed as trans-crocin-4).

Picrocrocin content (HPLC-CAD countergradient)

The content of picrocrocin was obtained by external calibration from the trans-crocin-4 standard used to quantify the content of crocins.

The content of picrocrocin in the extract thus obtained is 13.5%.

Safranal content (HPLC/DAD, λ=314 nm)

The content of safranal in the extract thus obtained is 0.06%.

Content of Total Polyphenols

The content of total polyphenols was obtained by the Folin spectrophotometric method. It is 4.6 g/100 g expressed as gallic acid equivalent.

### Example 3—Preparation of an Extract of Pistils of Crocus sativus Originating from the Taliouine Region Comprising 12% of Crocins (Outside of the Invention)

Saffron, in the form of dried whole pistils of Crocus Sativus flowers originating from the Taliouine region, supplied by Tresors et Saveurs, is ground with a cutting mill on a 1 mm screen. 1.5 kg of the orangey powder thus obtained is extracted under stirring in the absence of light with 15 litres of an ethanol-water mixture (1/1) at 25° C. for 4 h. The mixture is prefiltered on a 50 μm gauze in a closed filter. The spent biomass is emptied, re-extracted and pre-filtered according to the same protocol. The pre-filtrates from the two macerations are combined, pre-clarified in the presence of cellulose (Vitacel LC200) in a closed filter equipped with a 8-3.5 μm cellulose sheet then clarified in a bell-shaped filter equipped with a 0.8-0.5 μm cellulose sheet and finally sterilized using a 0.2 μm filter cartridge.

The sterilized aqueous-alcoholic filtrate is then evaporated under vacuum to remove the ethanol, and the slightly opaque aqueous solution is spray-dried to obtain the saffron extract in the form of a fine, orangey powder.

Equipment:
Grinding: Retsch SM100 cutting mill
Extraction: VSA 20E reactor
Pre-filtration: 400 mm closed filter with 50 μm Nitex Nylon gauze
Pre-clarification: 400 mm closed filter with FGC7 sheet
Clarification: 400 mm bell-shaped filter with FGC15 sheet
Sterilization: 0.2 μm filter cartridge
Concentrating: VSA20E reactor
Spray drying: Büchi Mini Spray Dryer B290

The specifications of the extract thus obtained are presented in Table 3 below.

TABLE 3

| | |
|---|---|
| bis-gentiobiosyl-E-crocetin/gentiobiosyl- | 59/26.5/9/5.5 |
| glucosyl-E-crocetin/bis-glucosyl-E- | (UV detection λ = |
| crocetin/bis-gentiobiosyl-13-Z-crocetin ratio | 440 nm) |
| Content of total crocin (expressed as bis- | 12.2% |
| gentiobiosyl-E-crocetin TC4) | |
| Safranal content | 0% |
| Picrocrocin content | 8.1% |
| Content of total polyphenols (expressed as | 4.1% |
| gallic acid equivalent) | |
| Water content | 5.7% |

TC4: trans-crocin-4.

Content of Total Crocin (HPLC/DAD, λ=440 nm)

Since the standards of the various crocins are not available, the quantification of the total crocin and of the various crocins (TC4+TC3+TC2+CC4) was carried out by external calibration relative to trans-crocin-4.

The results are therefore expressed as trans-crocin-4. trans-crocin-4 standard: Chengdu Biopurify Phytochemicals Ltd, ref. BP0406, purity 98%. The linearity of the trans-crocin-4 was verified between 0.5 and 100 μg/mL.

The content of total crocin in the extract thus obtained is 12.2% (expressed as trans-crocin-4).

Picrocrocin Content (HPLC-CAD Countergradient)

The content of picrocrocin was obtained by external calibration from a Santa Cruz reference SC-47934/SB batch E0317 picrocrocin standard.

The purity of the standard was estimated at 74% from the HPLC-CAD profile.

The linearity of the picrocrocin was verified between 5 and 200 μg/mL.

The content of picrocrocin in the extract thus obtained is 8.1%. The content of picrocrocin was obtained by external calibration from the trans-crocin-4 standard used to quantify the content of crocins.

Safranal Content (HPLC/DAD, λ=314 nm)

The content of safranal in the extract thus obtained is 0% (not detected).

Content of Total Polyphenols

The content of total polyphenols was obtained by the Folin spectrophotometric method. It is 4.1 g/100 g expressed as gallic acid equivalent.

### Example 4—Evaluation of the Antioxidant Activity of the Extract Obtained According to Example 2 (According to the Invention) Relative to the Extract Obtained According to Example 3 (Outside the Invention)

Study of Squalene Peroxidation

Protocol: The principle of the test is to determine, via the assaying of squalene (Sq) and its photo-oxidation product, the efficacy of an active agent against this form of photo-peroxidation.

The test makes use of squalene in the presence of a photosensitizer, hematoporphyrin. Under the action of UVA rays, singlet oxygen (¹O₂) is generated. This highly reactive form of oxygen degrades the squalene by oxidation of the double bonds to form squalene hydroperoxides (Sq-OOH).

In the context of the test, the exposure of squalene combined with hematoporphyrin is carried out in the presence of various concentrations of extract obtained according to Example 2 (according to the invention) and according to Example 3 (outside the invention). The reaction medium is ethanol.

Equipment and Reagents:
Squalene [Sigma S-3626]
Hematoporphyrine Free Base (approx. 70%) [Sigma—ref H-7253]
HPLC or UPLC system in reverse phase mode, detection in UV mode (205 nm)

Procedure:
ethanol solution containing 500 μg/ml of Sq and 2 μg/ml hematoporphyrin+extract to be tested
expose to UVA rays for the dose of 5 J/cm²
measure the amount of Sq and SqOOH before and after exposure
plot % inhibition as a function of the amount of extract tested. Deduce therefrom, where appropriate, IC25 or IC50 (amount of extract needed to reduce the squalene oxidation reaction by 25% or 50%).

$$\% inhibition = {( \frac{( \frac{SQOOH}{{SQ}_{résiduel}} essai - \frac{SQOOH}{{SQ}_{résiduel}} témoin )}{\frac{SQOOH}{{SQ}_{résiduel}} témoin} )}_{à5 Joules / cm 2} \times 100 \qquad [ Math . 1 ]$$

The results are given in table 3 below.

TABLE 4

| | Concentration | | |
|---|---|---|---|
| | in the reaction | Activity | |
| Starting materials | medium | (% inhibition) | Comments |
| Extract obtained | 10 ppm | 17% | IC25 = 19 ppm |
| according to | 26 ppm | 30% | IC50 = 56 ppm |
| Example 2 | 104 ppm | 66% | |
| (according to the | | | |
| invention) - solution | | | |
| in ethanol | | | |
| Extract obtained | 240 ppm | 78% | IC50 = 105 ppm |
| according to | | | |
| Example 3 (outside | | | |
| the invention) - | | | |
| solution in ethanol | | | |

The extract obtained according to Example 2 (according to the invention) as a high activity, with determination of an IC25=19 ppm and an IC50=56 ppm. This extract is therefore more effective than the extract obtained according to Example 3 (outside the invention) which has a much higher IC50=105 ppm.

Moreover, the extract obtained according to Example 2 (according to the invention) is more effective than vitamin E, which has an IC50 of the order of 0.025% (=250 ppm).

*IC25: concentration of raw material needed to inhibit squalene peroxidation by 25%.

**IC50: concentration of raw material needed to inhibit squalene peroxidation by 50%.

### Example 5—Olfactory Evaluation of the Extract Obtained According to Example 2 (According to the Invention) Relative to the Extract Obtained According to Example 2′ (Outside the Invention)

Extract 2 (according to the invention) and extract 2′ enriched with pure safranal to attain 0.10% of safranal (outside the invention) were evaluated olfactorily by a panel of 4 people.

The olfactory characteristics of the extract 2′ enriched to attain 0.10% of safranal (outside the invention): more powerful, warm, amber-like, woody, spicy, dirty note, cacao.

The olfactory characteristics of the extract 2 (according to the invention): less powerful, more fruity, honey-like, bread, green vegetable note.

It should be noted that, aside from the power:
amber-like, woody belong to the base notes, which are the most persistent/least volatile.
spicy, fruity belong to the heart notes.
green vegetable belongs to the head notes, which are the most volatile.

Thus, the extract 2 according to the invention has a less powerful odour than the extract 2′ outside the invention.

### Example 6—Face Cream Comprising the Extract Obtained According to Example 1 (According to the Invention)

TABLE 5

| | |
|---|---|
| Powdered potassium sorbate | 0.1% |
| Xanthan:polysaccharides:glucose/mannan/glucuronic acid | 0.3% |
| (40/30/30) | |
| Mixture of plants containing lecithin, fatty acids and | 5% |
| fatty alcohols | |
| Saffron extract obtained according to Example 1 | 0.1% |
| First cold-pressed organic sunflower oil | 20% |
| Glyceryl stearate citrate | 2% |
| Benzyl alcohol (and) dehydroacetic acid (and) water | 0.8% |
| Fragrance | 0.45% |
| Citric acid | 0.1% |
| Water (qs) | 100% |

1. Extract of at least one plant of the Crocus sativus species comprising (i) at least 20% by dry weight of one of more crocin(s) relative to the total weight of the components of a dry extract and (ii) less than 0.08% by dry weight of safranal relative to the total weight of the components of the dry extract.

2. Extract according to claim 1, in which said crocin(s) (i) is(are) present at between 20% and 40% by dry weight relative to the total weight of the components of the dry extract.

3. Extract according to claim 1, in which said safranal (ii) is present at less than 0.07% by dry weight relative to the total weight of the components of the dry extract.

4. Extract according to claim 1, in which the crocin(s) is(are) chosen from:
bis-gentiobiosyl-E-crocetin;
gentiobiosyl-glucosyl-E-crocetin;
bis-glucosyl-E-crocetin;
bis-gentiobiosyl-13-Z-crocetin;
gentiobiosyl-glucosyl-13-Z-crocetin;
and mixtures thereof.

5. Extract according to claim 1, in which the crocin(s) is(are) in the form of a mixture comprising:
between 10% and 30% by dry weight of bis-gentiobiosyl-E-crocetin;
between 5% and 10% by dry weight of gentiobiosyl-glucosyl-E-crocetin;
between 0.1% and 5% by dry weight of bis-glucosyl-E-crocetin; and
between 0.05% and 2% by dry weight of bis-gentiobiosyl-13-Z-crocetin;
relative to the total weight of the components of the dry extract.

6. Extract according to claim 1, in which the crocin(s) is(are) in the form of a mixture comprising:
between 60% and 70% by dry weight of bis-gentiobiosyl-E-crocetin;
between 20% and 30% by dry weight of gentiobiosyl-glucosyl-E-crocetin;
between 5% and 10% by dry weight of bis-glucosyl-E-crocetin; and
between 2% and 6% by dry weight of bis-gentiobiosyl-13-Z-crocetin;
relative to the total weight of the crocins of the dry extract.

7. Extract according to claim 1, in which said plant of the Crocus sativus species originates from Morocco.

8. Composition comprising, in a physiologically acceptable medium, an extract according to claim 1.

9. Composition according to claim 8, in which said extract is present in the composition in a concentration of between 0.0001% and 20% by weight relative to the total weight of the composition.

10. Cosmetic treatment method comprising the application to skin of the extract according to claim 1 to treat skin disorders induced by oxidative stress.

11. Cosmetic treatment method comprising the application to skin of the extract according to claim 1 to treat the signs of skin ageing.

12. Cosmetic treatment method comprising the application to skin of the extract according to claim 1 to as an antioxidant.

13. Cosmetic treatment method comprising the application to skin of the extract according to claim 1 to treat skin disorders induced by UV rays and/or pollution.

14. Cosmetic use of the extract according to claim 1 to treat the signs of skin ageing.

15. Process for preparing an extract of at least one plant of the Crocus sativus species according to claim 1 comprising extracting a part of at least one plant of the Crocus sativus species using an aqueous solvent chosen from water or a mixture of water and one or more water-miscible organic solvents.

16. Process according to claim 15, in which the extract obtained is subjected to the following:
i. filtration, and/or
ii. slurrying in cellulose, and/or
iii. one or more clarification steps, and/or
iv. sterilization.

17. Process according to claim 15, in which, on conclusion of said extraction, said extract is subjected to a drying step which comprises the removal of one(of the) solvent(s).
